(19) United States
(12) Patent Application Publication
(10) Pub. No.: US 2004/0224401 A1
(43) Pub. Date: Nov. 11, 2004

(54) Physiochemical culture conditions for embryonic stem cells

(76) Inventors: Tenneille E. Ludwig, Madison, WI (US); James A. Thomson, Madison, WI (US)

Correspondence Address: QUARLES & BRADY LLP, FIRSTAR PLAZA, ONE SOUTH PINCKNEY STREET, P.O. BOX 2113 SUITE 600, MADISON, WI, 53701-2113, US

(21) Appl. No.: 10/811,423

(22) Filed: Mar. 26, 2004

Related U.S. Application Data
(60) Provisional application No. 60/458,815, filed on Mar. 28, 2003.

(51) Int. Cl. C12N 5/08

(52) U.S. Cl. 435/366

(57) ABSTRACT

Physiochemical parameters to improve the culturing and sub-culturing (here called cloning) of human embryonic stem cells have been investigated. Low levels of oxygen and higher than expected levels of osmolarity in the culture medium have both been found to contribute to the improved culture of human stem cells.

### CROSS-REFERENCE TO RELATED APPLICATIONS

This application claims priority from U.S. provisional patent application Ser. No. 60/458,815 filed Mar. 28, 2003.

### STATEMENT REGARDING FEDERALLY SPONSORED RESEARCH OR DEVELOPMENT

This invention was made with United States government support awarded by the following agencies: NIH RR17721. The United States has certain rights in this invention.

### BACKGROUND OF THE INVENTION

Stem cells are cells which can be maintained in culture in vitro and which are capable of differentiation into many, if not all, of the differentiated cell types of a mature body. Stem cells are referred to as pluripotent which means that they are capable of differentiating into many differentiated cell types. One category of pluripotent stem cell of high interest is the human embryonic stem cell, which is a category of stem cell originally created from human embryos. Human embryonic stem cells are capable of indefinite proliferation in culture, are demonstrably pluripotent, and are probably totipotent. One potential use for human embryonic stem cells is to direct differentiation of stem cells into specific differentiation lineages to create differentiated cells or tissues for potential transplantation into human bodies for therapeutic purposes.

The techniques to create in culture human embryonic stem cells have been described, are replicable and do work, but efforts to refine many of the procedures are still appropriate. Human embryonic stem cells will proliferate in culture indefinitely, but the proliferation in the culture is relatively variable and subject to undesired differentiation in culture. It can take significant amounts of time to proliferate stem cells to make desired quantities for particular scientific experiments or treatments, and so optimizations which increase the culture or proliferation efficiency of the stem cell cultures would be useful. In short, the fully optimized conditions for the culture and proliferation of human embryonic stem cells have not yet been developed. There are several reasons why the development of standard and optimized conditions for the culture and proliferation of human embryonic stem cells would be desirable. One is simply to shorten the time necessary for the proliferation of undifferentiated stem cell cultures so that more stem cells can be created more easily. Another reason is to create well documented and standardized techniques so that different laboratories culturing stem cells can use common procedures and conditions and thus can obtain similar results. Yet another reason is the potential ultimate therapeutic use of such stem cells. To the extent that ultimate transplantation of cells or tissues derived from stem cells into human beings is an objective, the standardization and characterization of all of the components of the culture system from the beginning to the end of culture is an inherently desirable attribute.

### BRIEF SUMMARY OF THE INVENTION

The present invention is summarized in that a culture system has been developed for the culturing of human embryonic stem cells. The culture condition includes culture of the cells in an atmosphere having minimal oxygen, and may include the use of an antioxidant. The culture conditions for a human embryonic stem cell culture may also have an osmolarity in excess of 300 mOsMol.

It is an object of the present invention to define culture conditions which aid in the optimum growth of human embryonic stem cells.

It is a feature of the present invention that, surprisingly, the optimal conditions for the culture of human embryonic stem cells differ from the physiological conditions present in the human body.

Other objects, advantages and features of the present invention will become apparent from the following specification when taken in conjunction with the accompanying drawings.

### BRIEF DESCRIPTION OF THE SEVERAL VIEWS OF THE DRAWINGS

FIGS. 1 through 4 present graphical representations of data from the experimental work described below.

### DETAILED DESCRIPTION OF THE INVENTION

This specification is directed to improvements in systems for the optimized culture of human embryonic stem cells. It has heretofore been presumed that human embryonic stem cells would best be cultured in conditions which mimic the conditions found in somatic cells in vivo in a human body. This assumption turns out to be incorrect. It is described herein that by reducing the exposure of the human embryonic stem cells to oxygen in general, and oxygen free radicals in particular, and by increasing the osmolarity of the medium in which the stem cells are cultured beyond physiological levels, that the culture of human embryonic stem cells can be facilitated and encouraged. In other words, surprisingly, culture conditions which mimic the physiochemical conditions of the human body are not the conditions most optimal for the culture of human embryonic stem cells.

Two parameters which have been found to be important in the culture of stem cells, and two parameters which are different than what might have been expected, are the level of oxygen concentration in the atmosphere in which the cells are cultured and the osmolarity of the culture medium itself in which the stem cells live. Conventionally, mammalian cells in culture are cultured in an atmosphere containing both oxygen and carbon dioxide, generally with oxygen at ambient air concentrations. With regard to the oxygen concentration, it has been found here that stem cells grow better in culture and that the cloning efficiency of human stem cells is increased significantly, both under conditions of low oxygen. Cloning is used here to refer to the process of sub-culturing stem cells, or, in other words, the process by which a cell, or a very few cells, are taken out of one stem cell culture and introduced into a new culture vessel to start a new culture of stem cells. In its ideal practice, the cloning of a stem cell culture should result in a daughter culture of cells all derived from a single parental stem cell. Cloning efficiency refers to the relative degree of success and abundance of undifferentiated cells in the stem cell culture in the new culture vessel. Under poor cloning conditions, daughter culture can either fail to propagate or can propagate as differentiated cells, thereby losing the attribute of being stem cells. It is preferred for stem cell culture in general, and in particular for purposes of creating cultures which can be efficiently cloned, that the oxygen level be held to less than ambient atmospheric levels and preferably to about 5% or less of the content of the atmosphere to which the stem cell culture is exposed. It is also preferred that an antioxidant be added to the culture medium, to further decrease the level of oxygen free radicals in the culture medium. Many compounds having antioxidant effects are known. The addition of an antioxidant will, it is believed, act to lower the overall mutation rate of the stem cells in culture and will thus permit the cloning of undifferentiated stem cells with a lower level of mutation and differentiation than would otherwise be the case in comparable cultures without antioxidants added.

The osmolarity of the culture is another factor affecting the success and vitality of stem cell cultures. Osmolarity, measured in milli-osmoles, is a measure of the number of dissolved particles in a solution, which is a measure of the osmotic pressure that a solution will generate. Normal human serum has an osmolarity of about 290 milli-osmoles or mOsMol. Media for in vitro culture of other mammalian cells vary in osmolarity, but some media have an osmolarity as high as 330 mOsMol. Surprisingly, it has been found here that human embryonic stem cells grow best in an osmolarity of above 330 and preferably about 350 mOsMol. Osmolarity is adjusted in a medium for stem cell culture most simply by adjusting the concentration of salts, particularly NaCl, in the culture medium to achieve the osmolarity desired. There are any number of other salts that could be added to a medium to increase its osmolarity. The osmolarity of a solution can be measured by suitable instruments and can be calculated by the volume of the solution if one knows the number of molecules of salts which have been added.

While human embryonic stem cells can be grown in a number of culture media, the most common medium used is referred to as DMEM/DF12. This nomenclature indicates that the medium is a mixture, typically 50% each, of Dulbecco's modified Eagle Medium and Ham's F12 medium. DMEM and Ham's F12 are each media which are commercially available from many sources, separately or combined, and are specific combination of salts, vitamins, glucose, and amino acids. The first human stem cell cultures also included serum in the culture medium, but it has been since found that a serum replacement product may successfully be used to substitute for serum in the culture medium. Serum replacements, which contain purified albumin, vitamins, minerals, antioxidants, insulin, transferring and lipids, are available commercially or can be formulated originally from these ingredients. A suitable medium for stem cells culture is 80% DMEM/DF12 basal medium and 20% serum replacement, to which is also added glutamine, β-mercaptoethanol, non-essential amino acids, and a fibroblast growth factor. These constituents are all known previously and described in the art and may be used with the alterations discussed in this document.

### EXAMPLES

Effect of Gas Mixtures on Stem Cell Culture and Cloning

This investigation was to evaluate the effects of O₂ and CO₂ atmosphere on the ability of human embryonic stem cells to proliferate and remain undifferentiated. Cells were cultured in modified DMEM/DF12 plus “Knock-out” (Trademark) Serum Replacement from Gibco. The modified media included an adjustment to the sodium bicarbonate and sodium chloride concentrations to moderate pH and osmolarity. All media were corrected for appropriate pH given the concentration of CO₂, and the osmolarity was adjusted to also remain constant. Ultimate pH and osmolarity were constant across all media tested, despite alteration of atmospheric conditions. All media were conditioned overnight at standard atmospheric conditions (5% CO₂ in air) on mouse embryonic fibroblasts (MEFs) plated at a density of 2.12×10⁵ cells/ml prior to experimental use. Conditioning of the medium for stem cell culture is done to induce the stem cells to remain undifferentiated without exposing the cells to the MEFs themselves. Conditioning means the medium is used to culture MEFs before the medium is used to culture stem cells. Although the MEFs are removed from the conditioned medium prior to introduction of the stem cells, the medium is conditioned in some poorly understood manner and supports culture of undifferentiated stem cells in a manner that unconditioned media do not.

The human stem cells were individualized by treatment with trypsin, counted and plated onto six well plates. The human ES cells had been previously transformed with a green fluorescent protein (GFP) reporter gene under the control of an endogenous Oct4 promoter. Oct4 is known to be a marker of continued undifferentiated status. For the cell sorter analysis, 10K cells per well were plated. For cloning efficiency tests, 4K cells per well were plated. The treatments were all run in triplicate. The cells were fed and the atmosphere changed daily during the growth phase of the assay. FACS and cloning efficiency (CE) data were collected 8 days after plating the cells. The ratios of the total cell number and the geometric mean of GFP fluorescence intensity, compared to a control medium, were determined for each experimental treatment. Multiplication of the cell number ratio and the geometric mean ratio results in a media quality index (MQI) that is assigned to each experimental treatment. An MQI greater than 1 indicated an improvement over control conditions. Through this method, we were able to detect subtle differences due to physiochemical environment and media compositions that might not be apparent by subjective analysis.

The experimental conditions for atmospheric conditions were:

| | |
|---|---|
| 1. | 5% CO₂, 5% O₂, balance N₂ |
| 2. | 5% CO₂, 10% O₂, balance N₂ |
| 3. | 5% CO₂, balance air |
| 4. | 10% CO₂, 5% O₂, balance N₂ |
| 5. | 10% CO₂, 10% O₂, balance N₂ |
| 6. | 10% CO₂ in air |

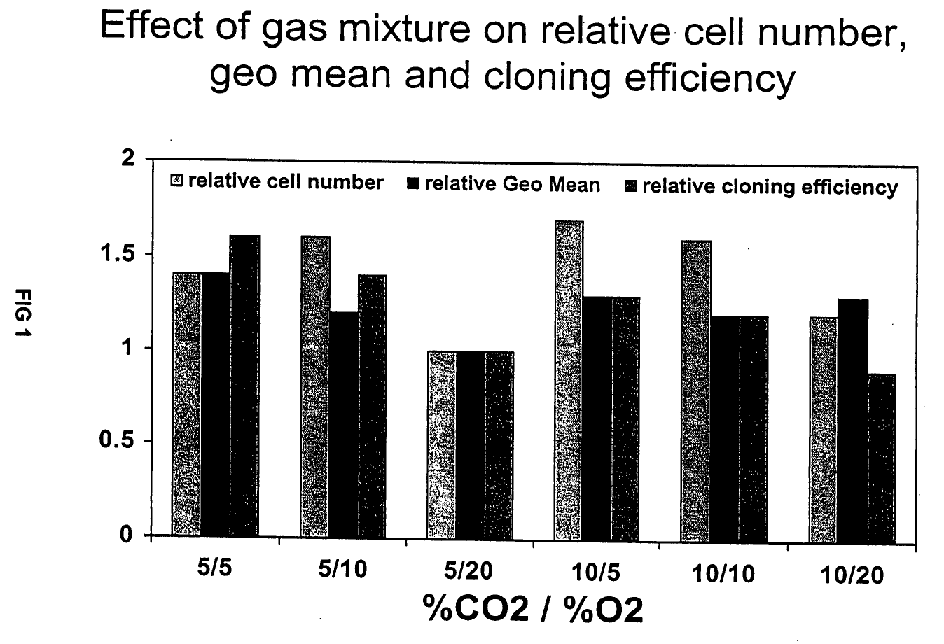

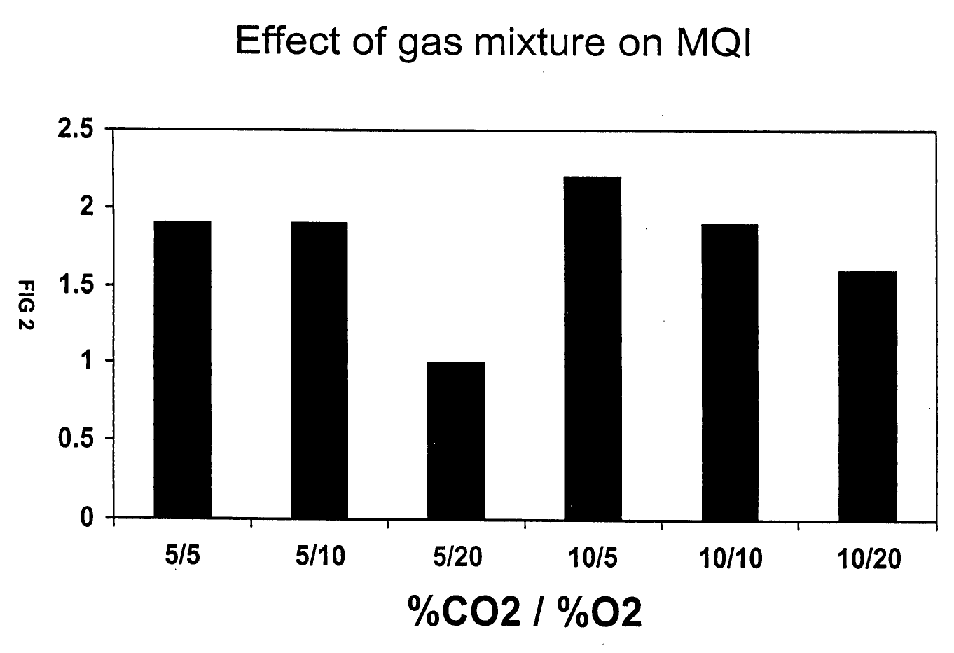

The results are summarized in the histograms FIGS. 1 and 2. In FIG. 1, the three bars for each experimental condition illustrate relative cell numbers, relative geometric mean of fluorescence detected, and relative cloning efficiency. FIG. 2 presents the data on MQI. Note that under any of the analytical metrics measured, the cell cultures that were exposed to less oxygen did better than those exposed to higher levels of oxygen. Thus culture with a lowered oxygen level results in improved stem cell growth in culture and increased cloning efficiency.

An additional experiment was performed adding a media supplement containing antioxidants. The data from this experiment suggested that addition of antioxidants will increase the attachment of stem cells and decrease the rate of differentiation.

Effect of Osmolarity

This investigation was to evaluate the effects of osmolarity on the ability of human embryonic stem cells to proliferate and remain undifferentiated. Human stem cells were again cultured in modified DMEM/DF12 plus “Knock-out” (trademark) Serum Replacement from Gibco. The osmolarity was adjusted to the levels set forth below by adjustment of the amount of sodium chloride in each medium. Media were all tested to assure that no alterations in pH or buffering capacity resulted from the adjustments in osmolarity. All media were again conditioned overnight on MEFs plated at a density of 2.12×10⁵ cells/ml prior to experimental use.

Again, the human embryonic stem cells were individualized by treatment with trypsin, counted and plated onto six well plates. The human ES cells had been previously transformed with a green fluorescent protein (GFP) reporter gene under the control of an endogenous Oct4 promoter, a marker of continued undifferentiated status. For the cell sorter analysis, 10K cells per well were plated. For cloning efficiency tests, 4K cells per well were plated. The treatments were all run in triplicate. The cells were fed daily during the growth phase of the assay. FACS and cloning efficiency (CE) data were collected 8 days after plating the cells. The same metrics of relative cell number, geometric mean of cell number, and MQI were calculated as were calculated for the experiments already described above.

The experimental conditions were the following:

| | |
|---|---|
| 1. | DMEM/F12 270 mOsMol |
| 2. | DMEM/F12 290 mOsMol |
| 3. | DMEM/F12 310 mOsMol |
| 4. | DMEM/F12 330 mOsMol |
| 5. | DMEM/F12 350 mOsMol |
| 6. | DMEM/F12 370 mOsMol |

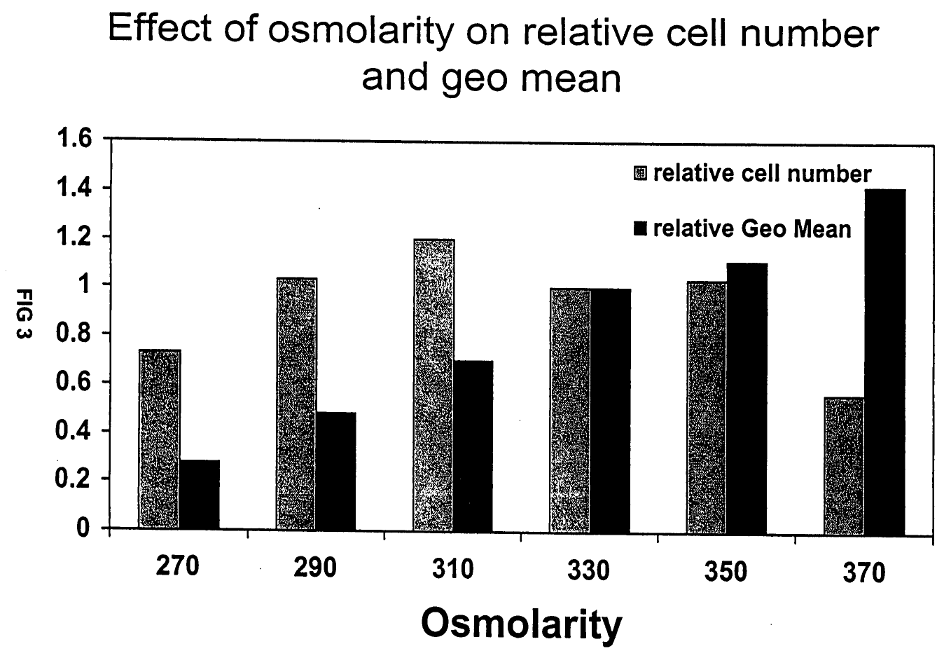

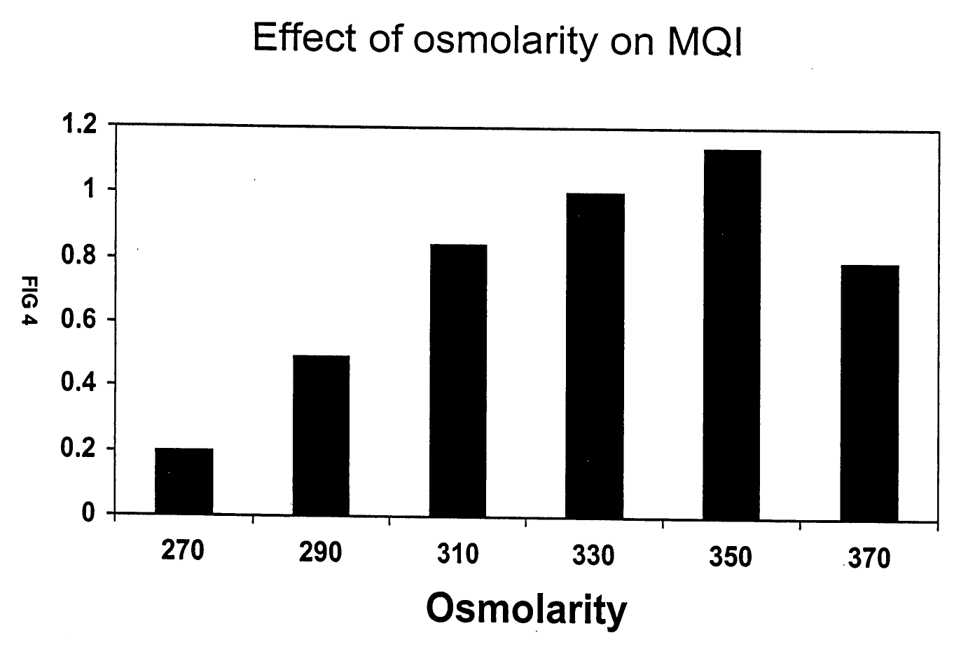

The results are displayed in FIGS. 3 and 4. FIG. 3 illustrates the relative cell number and geometric mean results, while FIG. 4 demonstrates the MQI results measured.

Note that the results, particularly the MQI, demonstrates that an osmolarity in excess of 330 mOsMol, and preferable an osmolarity of about 350 mOsMol is most efficient for stem cell culture. This result is surprising given the physiological conditions (290 mOsMol) of normal human serum.

We claim:

1. A method for culturing human embryonic stem cells comprising culturing the stem cells in a nutrient medium in which the stem cells will remain undifferentiated and in an atmosphere having no more than about 5% oxygen.

2. The method of claim 1 wherein the medium further comprises an antioxidant in the nutrient medium.

3. An improvement in methods for cloning cultures of human embryonic stem cells, the improvement comprising culturing the human embryonic stem cells prior to cloning in a nutrient medium and in an atmosphere having no more than about 5% oxygen.

4. The improvement of claim 3 further comprising adding an antioxidant to the nutrient medium in which the stem cells are cultured.

5. A method for culturing human embryonic stem cells comprising culturing the stem cells in a nutrient medium in which the stem cells can remain undifferentiated and in an atmosphere having no more than about 5% oxygen.

6. An improvement in a medium for the cultivation of human embryonic stem cells, the improvement comprising that the medium is adjusted to have an osmolarity in excess of 330 mOsMol.

7. The improvement as claimed in claim 6 wherein the osmolarity of the medium is about 350 mOsMol.

8. A stem cell culture comprising
a culture plate;
a nutrient medium in the culture plate;
growing human embryonic stem cells in the medium; and
the medium having an osmolarity in excess of 330 mOsMol.

9. A stem cell culture as claimed in claim 8 wherein the osmolarity is about 350 mOsMol.

10. A method for culturing human embryonic stem cells comprising culturing the stem cells in a nutrient medium in which the stem cells will remain undifferentiated, the medium having an osmolarity in excess of 330 mOsMol.

11. A method as claimed in claim 10 wherein the medium has an osmolarity of about 350 mOsMol.
